(19) United States
(12) Patent Application Publication
(10) Pub. No.: US 2026/0283536 A1
(43) Pub. Date: Sep. 24, 2026

(54) COGNITIVE DYSFUNCTION PREDICTION SYSTEM BASED ON GASTROINTESTINAL ELECTRICAL SIGNAL AND CONSTRUCTION METHOD

(71) Applicant: West China Hospital of Sichuan University, Chengdu (CN)

(72) Inventors: Lei CHEN, Chengdu (CN); Baichuan LI, Chengdu (CN); Shuming JI, Chengdu (CN)

(73) Assignee: West China Hospital of Sichuan University, Chengdu (CN)

(21) Appl. No.: 19/132,808

(22) PCT Filed: Jun. 27, 2023
(86) PCT No.: PCT/CN2023/102611

§ 371 Date: Jun. 2, 2026

(30) Foreign Application Priority Data
Nov. 25, 2022 (CN) 202211502992.9

(51) Int. Cl. A61B 5/00; A61B 5/392; A61B 5/397; G16H 50/20

(52) U.S. Cl. CPC A61B 5/4088; A61B 5/392; A61B 5/397; A61B 5/7267; G16H 50/20

(57) ABSTRACT

The present invention relates to the field of disease prediction, and in particular to a cognitive impairment prediction system based on a gastrointestinal electrical signal, and a method for constructing the same. In one aspect, the present invention provides a cognitive impairment prediction system, comprising: a database configured to store data, wherein a type of the data comprises gastrointestinal electrical signal data and clinical data, and the gastrointestinal electrical signal data are postprandial gastrointestinal electrical signal data; wherein the postprandial gastrointestinal electrical signal data comprise postprandial gastric dominant frequency, postprandial gastric percentage of disturbed electrical rhythm, postprandial gastric dominant power ratio, and postprandial intestinal lead time difference; wherein the clinical data comprise age, body mass index, high-density lipoprotein, and low-density lipoprotein; a data acquisition module; a model training module; and a prediction module configured to predict a probability of a subject developing cognitive impairment. In another aspect, the present invention further provides a method for constructing the cognitive impairment prediction system.

### PRIORITY

This application claims the priority of Chinese invention patent application CN202211502992.9, titled “A cognitive impairment prediction system based on a gastrointestinal electrical signal, and a method for constructing the same”, filed on Nov. 25, 2022, the entire content of which is incorporated herein by reference.

### TECHNICAL FIELD

The present invention relates to the field of disease prediction, and in particular to a cognitive impairment prediction system based on a gastrointestinal electrical signal, and a method for constructing the same.

### BACKGROUND

Early detection of diseases is critical. It is generally recognized that the earlier a disease is diagnosed, the greater the likelihood of curing (or successfully managing) the disease and achieving a better prognosis for the patient. If early screening and treatment of diseases can be realized, further deterioration may be prevented or delayed, and treatment efficacy may be improved (e.g., prolonging life expectancy or enhancing quality of life of the patient).

In recent years, the number of patients with chronic neurological diseases in China has continued to rise. However, due to reasons such as inconspicuous early symptoms, patients' limited awareness, complicated examination procedures, and high costs, certain chronic neurological diseases (e.g., cognitive impairment, sleep disorders, anxiety, or depression) are often diagnosed only when they progress to stages that are more difficult to intervene in or treat. Therefore, it is necessary to conduct large-scale screening in target populations (e.g., middle-aged and elderly people) to achieve early disease detection. However, large-scale screening often requires processing large volumes of data and relies on manual analysis, making the overall process time-consuming, labor-intensive, and costly, with analysis results that are highly subjective, complex, and difficult to quantify. As such, large-scale screening of many diseases remains difficult to implement, especially for the early detection of cognitive impairment.

Cognitive impairment refers to symptoms or diseases involving incomplete or deficient cognitive function. It may involve one or more impairments in memory, learning, language, executive function, visuospatial ability, calculation, and reasoning/judgment, and can severely impact a patient's ability to perform daily activities or social/occupational functions. At certain stages of disease progression, cognitive impairment may even be accompanied by psychiatric, behavioral, or personality abnormalities (e.g., anxiety, depression, or disability), placing a heavy burden on families and society. Moreover, statistics indicate that 15% to 20% of patients with mild cognitive impairment progress to Alzheimer's disease annually.

In the prior art, the diagnosis of cognitive impairment-related diseases includes basic clinical symptom assessment, neuropsychological evaluation, imaging techniques such as computed tomography (CT), magnetic resonance imaging (MRI), and single-photon emission computed tomography (SPECT), as well as biological fluid tests such as routine bloodwork, cerebrospinal fluid analysis, and antibody testing. Specifically, patients or their family members typically seek medical attention only after noticeable signs of cognitive impairment are observed. During clinical consultations, physicians typically administer one or more paper-based assessment scales to preliminarily evaluate a patient's cognitive status and interpret the results in conjunction with other test findings in order to identify whether the patient is potentially developing cognitive impairment. As such, the overall process is complex and tedious, time-consuming, labor-intensive, inefficient, and highly dependent on the physician's personal experience. Additionally, patients may have limited awareness of cognitive impairment-related diseases and show poor compliance during diagnosis. Due to age-related decline in hearing, vision, comprehension, or lower educational levels, many patients are unable to independently communicate with the physician or complete the assessment scales, and thus rely on family members for assistance. These factors make early detection (especially large-scale screening in grassroots or broad populations), timely targeted treatment, and prevention more difficult.

Although Internet-based cognitive impairment screening or evaluation systems exist in the prior art, they primarily involve completing online assessment scales and remote consultations with physicians. These systems fail to address the problems of procedural complexity, time consumption, and labor intensity, and are particularly unsuitable for individuals with low educational level, limited comprehension, or unfamiliarity with digital devices, thereby restricting their practical application.

### SUMMARY

In a first aspect, the present invention provides a cognitive impairment prediction system based on a gastrointestinal electrical signal, comprising:
a database configured to store data, wherein a type of the data comprises gastrointestinal electrical signal data and clinical data, and the gastrointestinal electrical signal data are postprandial gastrointestinal electrical signal data; wherein the postprandial gastrointestinal electrical signal data comprise postprandial gastric dominant frequency, postprandial gastric percentage of disturbed electrical rhythm, postprandial gastric dominant power ratio, and postprandial intestinal lead time difference; wherein the clinical data comprise age, body mass index, high-density lipoprotein, and low-density lipoprotein; wherein the data comprise sample data from a sample population and subject data from a subject;
a data acquisition module configured to acquire the data and store the data in the database;
a model training module configured to train the sample data using a machine learning algorithm to establish a cognitive impairment prediction model;
a prediction module configured to acquire the subject data through the data acquisition module and analyze the subject data using the cognitive impairment prediction model to predict a probability of the subject developing cognitive impairment.

In some embodiments, the sample data are divided into a training set and a validation set in a ratio of 7:3.

In some embodiments, the gastrointestinal electrical signal data are simultaneously collected through leads respectively positioned at a gastric body, a gastric antrum, a lesser gastric curvature, a greater gastric curvature, an ascending colon, a transverse colon, a descending colon, and a rectum.

In some embodiments, the system further comprises a validation module configured to evaluate an accuracy of the cognitive impairment prediction model using the validation set.

In some embodiments, an evaluation indicator of the evaluation comprises one or more of calibration, discrimination, and clinical utility.

In some embodiments, the cognitive impairment is mild cognitive impairment.

In a second aspect, the present invention provides a method for constructing a cognitive impairment prediction system based on a gastrointestinal electrical signal, wherein the cognitive impairment prediction system comprises a cognitive impairment prediction model, the method comprises:
S1 acquiring sample data from a sample population, wherein a type of the sample data comprises gastrointestinal electrical signal data and clinical data;
S2 performing a pre-training on the sample data to select a predictive variable, wherein the selected predictive variable comprises postprandial gastric dominant frequency, postprandial gastric percentage of disturbed electrical rhythm, postprandial gastric dominant power ratio, postprandial intestinal lead time difference, age, body mass index, high-density lipoprotein, and low-density lipoprotein;
S3 based on the selected predictive variable, training the sample data using a machine learning algorithm to establish the cognitive impairment prediction model, thereby constructing the cognitive impairment prediction system based on a gastrointestinal electrical signal.

In some embodiments, the sample data are divided into a training set and a validation set in a ratio of 7:3.

In some embodiments, the pre-training comprises a first-round variable selection and a second-round variable selection; wherein the first-round variable selection comprises LASSO regression analysis, and the second-round variable selection comprises logistic regression analysis and stepwise regression analysis.

In some embodiments, the method further comprises S4 evaluating an accuracy of the cognitive impairment prediction model using the validation set, wherein an evaluation indicator of the evaluation comprises one or more of calibration, discrimination, and clinical utility.

In some embodiments, the pre-training comprises ridge regression or a random forest model.

Compared with the prior art, the present invention provides the following beneficial technical effects:

The present invention provides a cognitive impairment prediction system based on a gastrointestinal electrical signal, and a method for constructing the same. In the present invention, a total of 46 feature variables in the sample data are pre-trained (feature selection) using LASSO regression analysis, logistic regression analysis, and stepwise regression analysis. As a result, eight predictive variables are retained, namely: postprandial gastric dominant frequency, postprandial gastric percentage of disturbed electrical rhythm, postprandial gastric dominant power ratio, postprandial intestinal lead time difference, age, body mass index (BMI), high-density lipoprotein, and low-density lipoprotein. A cognitive impairment prediction model is then constructed based on these eight predictive variables.

In the prior art, cognitive impairment is typically diagnosed through the use of assessment scales, memory tests, and imaging techniques. Patients with mild cognitive impairment may exhibit declines in memory, language, and other cognitive functions, while daily and social functioning may remain relatively unaffected. However, the symptoms presented in the early stages are often subtle and easily overlooked, resulting in missed opportunities for timely intervention. By the time the patient or individuals around the patient recognize the potential onset of cognitive impairment, the condition may have already advanced to a stage less amenable to effective intervention and treatment or may have progressed to irreversible Alzheimer's disease. In contrast, based on the cognitive impairment prediction model, the cognitive impairment prediction system and method provided by the present invention requires only certain gastrointestinal electrical signal data and basic clinical data (e.g., age, BMI, high-density lipoprotein, and low-density lipoprotein) from the subject to predict the risk of developing cognitive impairment-without relying on complex testing procedures or assessment scales. This facilitates early screening and helps reduce the risk of progression to other diseases, such as Alzheimer's disease.

Furthermore, the cognitive impairment prediction system and method provided by the present invention do not require costly or potentially invasive testing procedures, such as biological fluid tests or imaging techniques, nor do they necessitate completion of assessment scales by the subject. Accordingly, the cognitive impairment prediction system and method provided by the present invention feature a simplified procedure, cost-effectiveness, and enhanced subject acceptance and compliance. The cognitive impairment prediction system and method provided by the present invention are not limited by the subject's age or educational background, and are not affected by communication or comprehension barriers during clinical consultations, nor by the physician's personal experience. Subject data can be analyzed and predicted in an objective and non-invasive manner. Therefore, the present invention is particularly well-suited for large-scale preliminary screening and early detection of cognitive impairment, such as in community settings or physical examination centers. In summary, the cognitive impairment prediction system and method provided by the present invention not only support clinical assessment but also facilitate individualized risk prediction and are widely applicable across various settings and populations, including primary care institutions, homes, hospitals, and physical examination centers.

### DESCRIPTION OF DRAWINGS

To make the embodiments of the present invention or the technical solutions in the prior art clearer, the drawings required to be used in the description of the embodiments or the prior art will be briefly introduced below. In all drawings, similar elements or components are generally identified by similar reference numerals. It is obvious that the drawings described below are some embodiments of the present invention, and that other drawings can be obtained from these drawings for those of ordinary skill in the art without making inventive effort.

FIG. 1A is a schematic diagram of the LASSO regression model according to Embodiment 1 of the present invention.

FIG. 1B is a schematic diagram of the ten-fold cross-validation results according to Embodiment 1 of the present invention.

FIG. 2A is a schematic diagram of the nomogram of the optimal logistic regression model according to Embodiment 1 of the present invention.

FIG. 2B is a schematic diagram of the dynamic nomogram of the optimal logistic regression model according to Embodiment 1 of the present invention.

FIG. 3A is a schematic diagram of the ROC curve of the prediction model of the test set according to Embodiment 1 of the present invention.

FIG. 3B is a schematic diagram of the ROC curve of the prediction model of the validation set according to Embodiment 1 of the present invention.

FIG. 4A is a schematic diagram of the calibration curve of the test set according to Embodiment 1 of the present invention.

FIG. 4B is a schematic diagram of the calibration curve of the validation set according to Embodiment 1 of the present invention.

FIG. 5A is a schematic diagram of the decision curve of the test set according to Embodiment 1 of the present invention.

FIG. 5B is a schematic diagram of the decision curve of the validation set according to Embodiment 1 of the present invention.

FIG. 6 is a schematic diagram showing the placement positions of leads in the embodiment of the present invention.

FIG. 7 is a schematic diagram of the architecture of the prediction system provided in the embodiment of the present invention.

FIG. 8 is a schematic diagram of the module of the prediction system provided in the embodiment of the present invention.

FIG. 9 is a diagram showing the summary of the 46 relevant feature variables included in Embodiment 1 of the present invention.

FIG. 10 is a diagram showing the results of the first-round variable selection in Embodiment 1 of the present invention.

FIG. 11 is a diagram showing the results of the second-round variable selection in Embodiment 1 of the present invention.

100—prediction system, 102—data acquisition module, 104—model construction module, 106—database, 108—model training module, 110—validation module, 112—prediction module, 202—first terminal, 204—second terminal, 206—network, 601—gastric body, 602—lesser gastric curvature, 603—greater gastric curvature, 604—antrum, 605—ascending colon, 606—transverse colon, 607—descending colon, 608—rectum.

### DETAILED DESCRIPTION

To make the objective, the technical solutions and advantages of the embodiments of the present invention clearer, the technical solutions of the embodiments of the present invention will be clearly and completely described below in combination with drawings. It is obvious that the described embodiments are some of the embodiments of the present invention, not all of the embodiments. Based on the embodiments of the present invention, all other embodiments obtained by those of ordinary skill in the art without making inventive effort shall belong to the protection scope of the present invention.

As used herein, the term “and/or” refers to any and all combinations of one or more of the associated listed items.

As used herein, “a plurality of” means two or more, including but not limited to two, three, four, five, and so on.

It should be noted that the term “include”, “comprise” or any variant thereof is intended to encompass nonexclusive inclusion so that a process, method, article or device including a series of elements includes not only those elements but also other elements which have been not listed definitely or an element(s) inherent to the process, method, article or device. Moreover, the expression “comprising a(n) . . . ” in which an element is defined will not preclude presence of an additional identical element(s) in a process, method, article or device comprising the defined element(s) unless further defined.

As used herein, the term “about”, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

### Embodiment 1: Gastrointestinal Electrical Signals and Cognitive Impairment Prediction Model

### 1.1 Method

Subjects: The study recruits community-dwelling individuals over 40 years of age from 60 communities in western China who voluntarily participate and provide informed consent. General information is collected for each subject, including age, gender, marital status, education level, lifestyle, and dietary habits. Each subject also undergoes the Mini-Mental State Examination (MMSE) and electrogastroenterography (EGEG) testing. Exclusion criteria include individuals with hearing or visual impairments; individuals diagnosed within the past six months with gastrointestinal diseases such as gastritis or gastric ulcers, or presenting with gastrointestinal uncomfortable symptoms such as diarrhea or constipation; individuals with severe dysfunction of major organs (e.g., heart, liver, kidney) or metabolic disorders such as diabetes; and individuals with major psychiatric disorders. In order to reduce the influence of medications on EGEG results, individuals who have taken any medications within one week prior to testing are also excluded.

Cognitive function assessment: The cognitive function of each subject is assessed using the Mini-Mental State Examination (MMSE), a tool that demonstrates established reliability and validity for detecting cognitive impairment.

Each subject completes the assessment under standardized verbal instructions in a quiet setting, with guidance provided by two trained professionals.

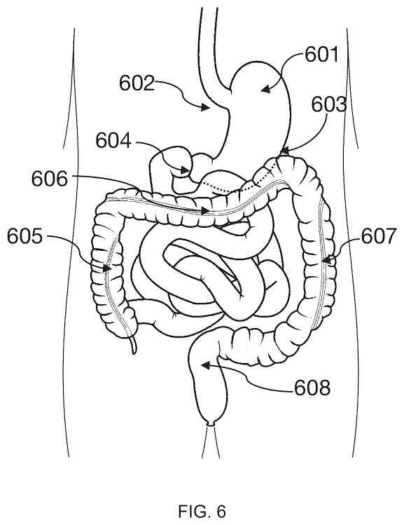

EGEG recording: An 8-channel electromyograph (XDJ-S8, Hefei Kaili Co., Hefei, China) is used to measure and collect myoelectric activity signals from the gastrointestinal tract. All subjects are instructed to avoid alcohol, spicy, or irritant foods for three days prior to the testing and to fast for at least six hours before testing. During the measurement, each subject is placed in a supine position. Four gastric electrodes (positioned at the gastric body 601, lesser gastric curvature 602, greater gastric curvature 603, and antrum 604, respectively) and four intestinal electrodes (positioned at the ascending colon 605, transverse colon 606, descending colon 607, and rectum 608, respectively) (Hanjie Co. Ltd., Shanghai, China) are applied to the abdominal skin, as shown in FIG. 6. Subjects are instructed to remain still and silent throughout the procedure. A 6-minute preprandial EGEG recording is first conducted. The subject then consumes approximately 200 kcal of standard food as part of a meal loading test, followed by a 6-minute postprandial EGEG recording. Electrode (lead, or channel) placements are as follows (see FIG. 6): gastric body 601: 3-5 cm to the left and 1 cm above the midpoint between the xiphoid process and the navel; antrum 604: 2-4 cm to the right of the midpoint between the xiphoid process and the navel; lesser gastric curvature 602: halfway above the midpoint between the xiphoid process and the navel; greater gastric curvature 603: halfway below the midpoint between the xiphoid process and the navel; ascending colon 605: 2-4 cm to the right of the navel; transverse colon 606: 1 cm below the navel; descending colon 607: 2-4 cm to the left of the navel; rectum 608: below the coccygeal region on the lower back.

Gastrointestinal electrical indicators: EGEG signals are recorded at a sampling frequency of 1 Hz and a filtering frequency range of 0.008-0.1 Hz to remove background noise, including interference from cardiac activity. Following artifact detection, the raw EGEG data are processed using the built-in software of the electromyography device. Through spectral analysis, the following parameters are respectively extracted for each of the above-mentioned eight leads: (1) mean amplitude of waveform; (2) mean frequency of waveform; (3) gastric (or intestinal) percentage of disturbed electrical rhythm; (4) reaction area of waveform; (5) lead time difference; (6) dominant frequency; (7) dominant power ratio; (8) percentage of normal slow wave; (9) coupling percentage.

Other indicators: Each subject undergoes blood testing to measure blood glucose and blood lipid indicators, including glucose, triglycerides, cholesterol, high-density lipoprotein (HDL), and low-density lipoprotein (LDL). Additionally, basic personal characteristics are collected, including gender, age, smoking history, alcohol drinking history, and body mass index (BMI).

Construction of prediction model: First, to obtain a subset of predictive factors, least absolute shrinkage and selection operator (LASSO) regression, a type of regularization algorithm—is performed in the first-round variable selection.

Furthermore, the LASSO regression analysis is run with 10-fold cross-validation for centralization and normalization of the included variables, and then “lambda. min” is used to select those with the best performance. Subjects are randomly divided into a training set and a validation set in a ratio of 7:3. Subsequently, stepwise multivariate logistic regression is performed in the second-round variable selection based on the predictive factors selected in the LASSO regression model. Statistically significant predictive factors (where the terms “predictive factor” and “predictive variable” are used interchangeably in the present invention) are retained for constructing the prediction model. Finally, the resulting prediction model is then applied to predict the risk of cognitive impairment and to construct a corresponding nomogram prediction model. It is to be understood that other suitable algorithms known in the art may also be employed, such as random forest, other regularization methods (e.g., ridge regression), or neural networks.

Additionally, the accuracy of the prediction model is evaluated using data from the training and validation sets through multiple validation methods, including: Receiver Operating Characteristic (ROC) curves, wherein the area under the curve (AUC) is used to evaluate the quality of the cognitive impairment risk nomogram in distinguishing true positives from false positives (i.e., discrimination); calibration curves, along with the Hosmer-Lemeshow test, are used to evaluate the calibration of the cognitive impairment risk nomogram; decision curves, wherein the decision curve analysis is used to determine the clinical utility of the cognitive impairment risk nomogram, based on net benefit under different threshold probabilities in a natural population cohort. All analyses are performed using R version 4.1.3 with packages glmnet and rms, and the significance level is set as a two-tailed a of <0.1.

### 1.2 Results

### Subject Data Information:

A total of 886 subjects complete all relevant assessments, including 273 males and 613 females. Among them, 106 subjects are diagnosed with mild cognitive impairment (MCI), including 71 males and 35 females. For each subject, the gastrointestinal electrical signal indicators are calculated as the average values derived from the postprandial or postprandial parameterized indicator data collected via the above-mentioned eight leads, representing preprandial electrical signal indicators of gastric leads, postprandial electrical signal indicators of gastric leads, preprandial electrical signal indicators of intestinal leads, and postprandial electrical signal indicators of intestinal leads, respectively. This method-simultaneously collecting signals from multiple leads at multiple anatomical locations and calculating the average-allows for better capture of the overall motility patterns of the stomach and intestines, thereby effectively obtaining signals that more accurately reflect the actual overall condition of the stomach and intestines. Additionally, preliminary experiment in the present invention demonstrates that the gastrointestinal electrical signal indicators obtained through this multipoint collection method are relatively stable, which facilitates model construction and enhances the generalizability of the constructed model for application in large-scale populations.

All subjects are randomly allocated into a training set and a validation set in a 7:3 ratio, resulting in 620 cases in the training set and 266 cases in the validation set. In both datasets, statistically significant differences (P<0.01) are observed between MCI and non-MCI subjects with respect to age, high-density lipoprotein (HDL), and postprandial gastric dominant frequency (stomach. main. frequency. aftermeal, SMFA).

### Selection of Independent Risk Factors:

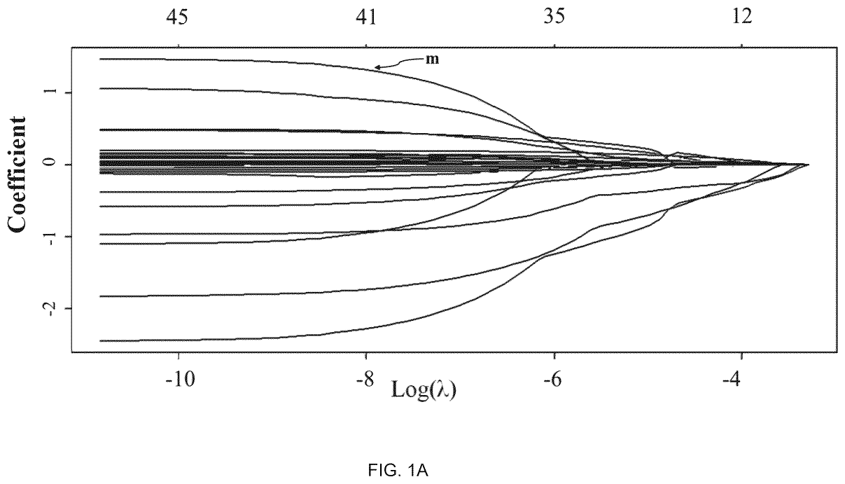

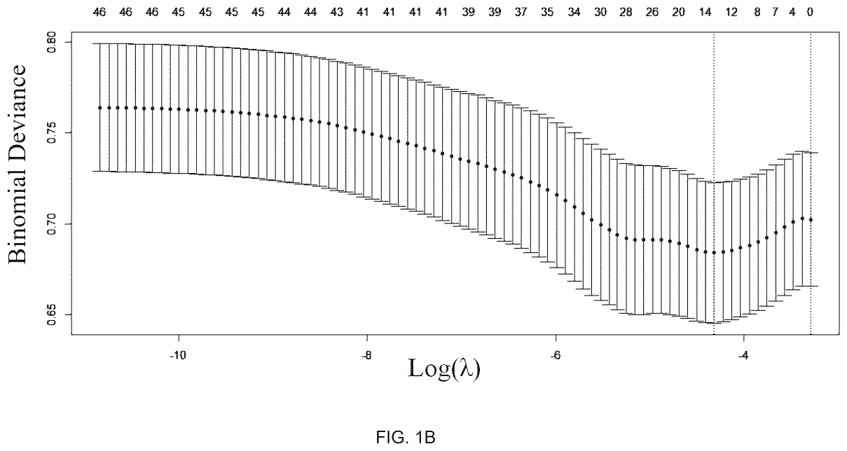

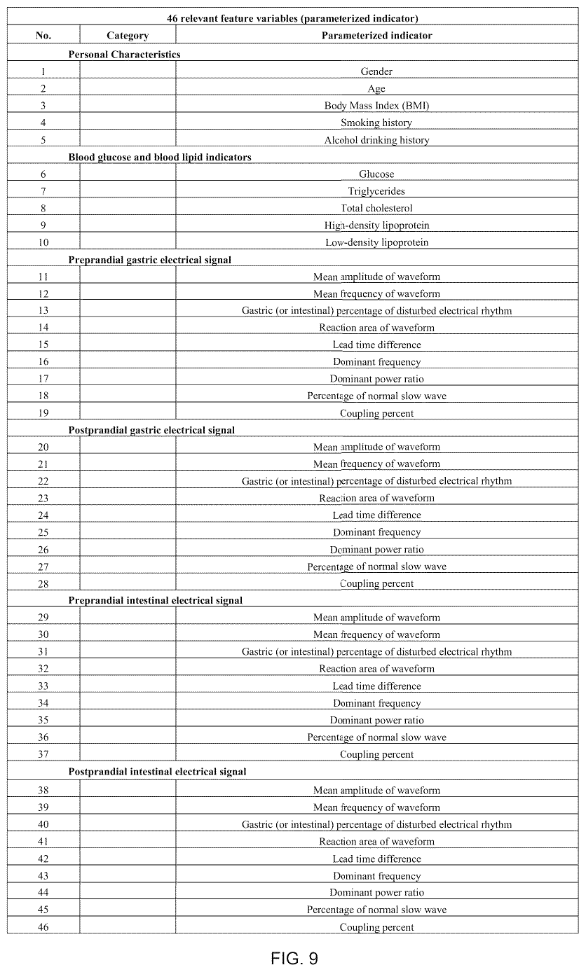

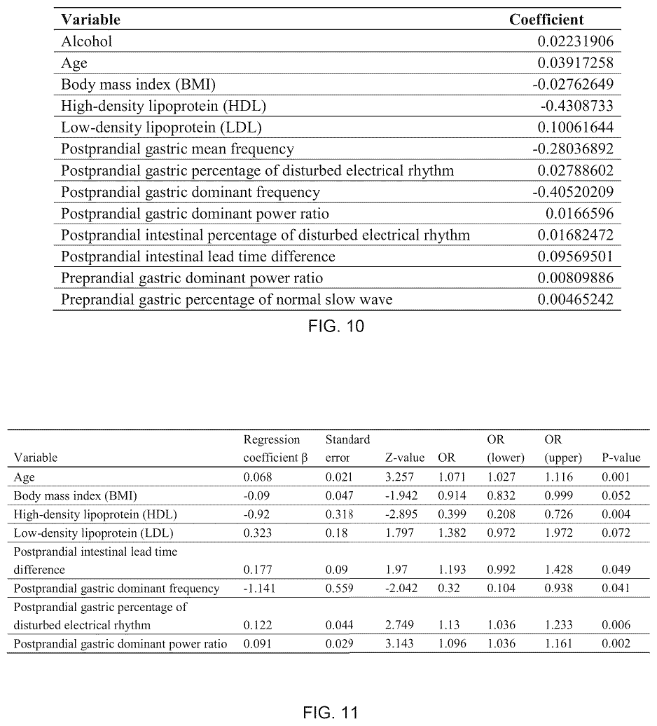

A feature variable selection method based on non-zero coefficients in LASSO regression is employed to screen predictive variables from a total of 46 relevant feature variables (also referred to as independent variables (IV) in the present invention) (see FIG. 9). In the technical solution of the present invention, 13 feature variables are ultimately retained as candidate predictive variables for the artificial intelligence model (see FIGS. 1A and 1B, described in detail below) used to predict the response variable (also referred to as the dependent variable (DV) in the present invention)—namely, the risk of cognitive impairment. The retained 13 feature variables include: alcohol, age, body mass index (BMI), high-density lipoprotein, low-density lipoprotein, postprandial gastric mean frequency, postprandial gastric dominant frequency, postprandial gastric percentage of disturbed electrical rhythm, postprandial gastric dominant power ratio, postprandial intestinal percentage of disturbed electrical rhythm, postprandial intestinal lead time difference, preprandial gastric dominant power ratio, and preprandial gastric percentage of normal slow wave (see FIG. 10).

The LASSO (Least Absolute Shrinkage and Selection Operator) regression analysis utilized in the technical solution of the present invention serves as a method for coefficient shrinkage and feature variable selection in linear regression models. In order to obtain a subset of predictive factors, the LASSO regression analysis imposes a penalty on the model parameters, thereby forcing the regression coefficients (also referred to as “coefficient” in FIG. 1A) of certain feature variables toward zero to minimize prediction error for the response variable.

Following this shrinkage process, feature variables with zero-valued coefficients are eliminated from the model, whereas those with non-zero coefficients are retained as being strongly correlated with the response variable. The parameter A governs the complexity of the LASSO regression model. Specifically, a larger A imposes a greater penalty on a linear regression model with more feature variables, resulting in a simpler model comprising fewer predictive variables, each of which exhibits a strong correlation with the response variable—thereby yielding a model with optimal predictive performance. As shown in FIG. 1A, each curve illustrates the trajectory of a feature variable's regression coefficient as λ changes. The vertical axis represents the coefficient value, the lower horizontal axis represents log(λ), and the upper horizontal axis represents the number of non-zero coefficients in the model. For example, the independent variable labeled “m” in FIG. 1A retains a non-zero coefficient even at higher λ values, with the coefficient value increasing as λ decreases. In other words, the first-round variable selection removes feature variables whose regression coefficients are prone to shrink to zero among the 46 relevant feature variables, and retains the above-mentioned 13 feature variables as the predictive variables for constructing the prediction model.

Furthermore, to more accurately evaluate the performance of the prediction model based on the above 13 predictive variables, the technical solution of the present invention employs LASSO regression analysis in combination with 10-fold cross-validation. The evaluation is performed based on the log-likelihood function (specifically, the −2 log-likelihood) and the binary dependent variable (also referred to as a “yes/no” type variable), with the objective of minimizing the target parameter during model selection through cross-validation. All 46 feature variables are centralized and normalized. The optimal value of the parameter λ (lambda) is then selected accordingly. As shown in FIG. 1B, due to cross-validation, for each λ value, the black dot represents the mean of the target parameter, and the solid line above and below the black dot represents the confidence interval of the target parameter. Two dashed lines indicate two specific λ values: Lambda. min and Lambda.1se. Any λ between these two values may be considered acceptable. A model constructed using Lambda.1se (i.e., the A value yielding the simplest model within one standard error of Lambda.min) is the simplest, including the fewest number of predictive variables. In contrast, a model constructed using Lambda.min (i.e., the λ value corresponding to the minimum mean target parameter across all λ values) achieves higher predictive accuracy. Therefore, the technical solution of the present invention adopts Lambda.min in order to construct a prediction model with optimal performance and the highest accuracy.

### Development of Prediction Model

In the technical solution of the present invention, stepwise multivariate logistic regression analysis is performed to construct the prediction model based on the predictive variables selected via the LASSO regression model. The selected predictive variables are then evaluated for statistical significance, and those determined to be statistically significant are retained as predictive variables (or predictive factors) for constructing the cognitive impairment risk prediction model.

The technical solution of the present invention further applies a logistic regression model to analyze the 13 previously selected predictive variables. Through stepwise regression, optimal predictive variables are selected, and 8 predictive variables are ultimately retained, each exhibiting statistical significance at the 0.1 level. These 8 predictive variables include: age, body mass index (BMI), high-density lipoprotein, low-density lipoprotein, postprandial gastric dominant frequency, postprandial gastric percentage of disturbed electrical rhythm, postprandial gastric dominant power ratio, and postprandial intestinal lead time difference (see FIG. 11). The technical solution of the present invention also tests these 8 predictive variables using various statistical methods, with particular emphasis on their odds ratios (ORs). The OR is a statistical measure used to quantify the strength of association between two events. It represents the ratio of the probability of an outcome occurring in the presence of a specific exposure (in the context of the present invention, the exposure refers to a tested predictive variable, hereinafter the same) to the probability of the outcome occurring in the absence of that exposure. In the present invention, the OR value may be considered as the strength of association between cognitive impairment (i.e., the response variable) and a specific exposure. It indicates how many times more likely individuals with the specific exposure are to develop cognitive impairment (also referred as the disease risk) compared to individuals without such exposure. If the OR value of a tested predictive variable>1, it suggests that the exposure increases the risk of cognitive impairment, indicating a positive association between the predictive variable and cognitive impairment. If the OR value of a tested predictive variable<1, it suggests that the exposure decreases the risk of cognitive impairment, indicating a negative association between the predictive variable and cognitive impairment. If the OR value of a tested predictive variable=1, it indicates no association between the predictive variable and cognitive impairment. The 95% confidence interval (CI) provides an estimate of the precision of the odds ratio (OR) obtained through statistical testing. It represents the range within which the true population value of the OR is expected to lie with 95% confidence. A narrower CI indicates higher precision and robustness of the OR obtained through statistical testing. In FIG. 11, “OR (upper)” and “OR (lower)” indicate the upper and lower bounds of the 95% CI, respectively, for each tested predictive variable. FIG. 11 illustrates the OR values and their corresponding 95% confidence intervals of the retained eight predictive variables, demonstrating that all of the predictive variables are associated with cognitive impairment and are therefore applied into the cognitive impairment risk prediction model of the present invention.

In addition, in FIG. 11, the regression coefficient β represents the partial regression coefficient of the predictive variable associated with the response variable, obtained through the logistic regression analysis. It represents both the magnitude and direction of the effect that a one-unit increase in a predictive variable has on the response variable. After standardization, the regression coefficients of different predictive variables become comparable. The regression coefficient β for each tested predictive variable is an estimated value. The standard error refers to the standard error of the regression coefficient β for each tested predictive variable and represents the precision of the regression coefficient. A larger standard error indicates lower precision of the tested predictive variable. The Z-value (Z statistic) is calculated by dividing the regression coefficient β of the tested predictive variable by its standard error and is primarily used to determine the P-value of the tested predictive variable. The P-value refers to the probability value corresponding to the Z statistic of each tested predictive variable. A smaller P-value indicates greater importance of the corresponding predictive variable with respect to the response variable.

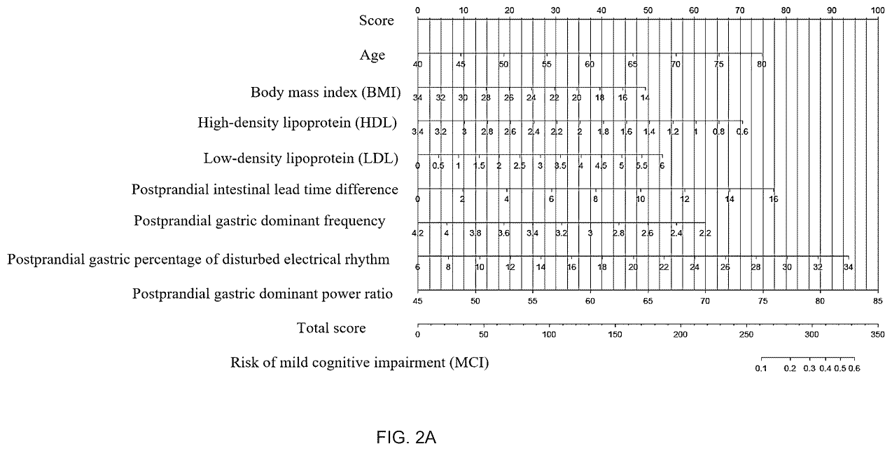

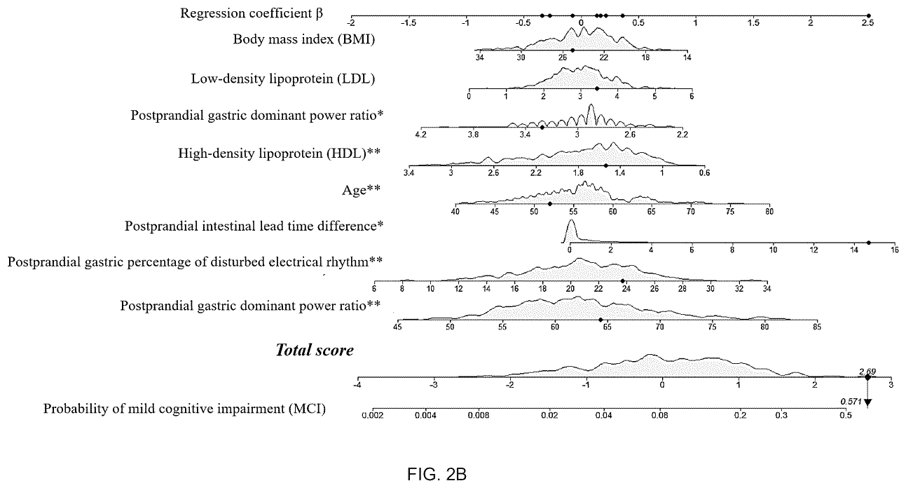

Based on the eight predictive variables described above, the present embodiment constructs a cognitive impairment risk prediction model. To facilitate visualization of the constructed model, a corresponding nomogram is generated, as shown in FIGS. 2A and 2B (in FIG. 2B, *P<0.05 is considered statistically significant, and **P<0.01 is considered highly significant). FIGS. 2A and 2B illustrate different formats of the nomogram representing the cognitive impairment prediction model of the present invention. In FIGS. 2A and 2B, each variable (e.g., “Age”, “Body mass index (BMI)”) is represented by a horizontal line labeled with scale marks indicating its value range. The length of each line indicates the relative contribution of that variable to the outcome event (i.e., the occurrence of cognitive impairment). For a given value, the individual score associated with each variable is read from the top of FIG. 2A or 2B, under “Score” or “Regression coefficient 3,” respectively. A total score is calculated by summing the individual scores corresponding to each variable. This total score corresponds to a predicted probability of mild cognitive impairment (MCI), which is shown at the bottom of the nomogram in FIG. 2A or 2B and labeled as “Risk of mild cognitive impairment (MCI)” or “Probability of mild cognitive impairment (MCI)”. For instance, in FIG. 2A, if a subject has a total score of 300, the predicted risk of developing MCI is 0.3 (i.e., 30%). In addition, in the dynamic nomogram shown in FIG. 2B, the black dot on each variable's corresponding line represents the actual value for a given subject, while the waveform above each line illustrates the distribution of that variable. From the position of the black dot, a corresponding individual score can be obtained from the “regression coefficient (” line above. A total score is calculated by summing the individual scores corresponding to each variable, which in turn yields the predicted probability of MCI. In the example illustrated in FIG. 2B, the subject has a predicted MCI risk of 0.571 (i.e., 57.1%).

### Validation of Prediction Model

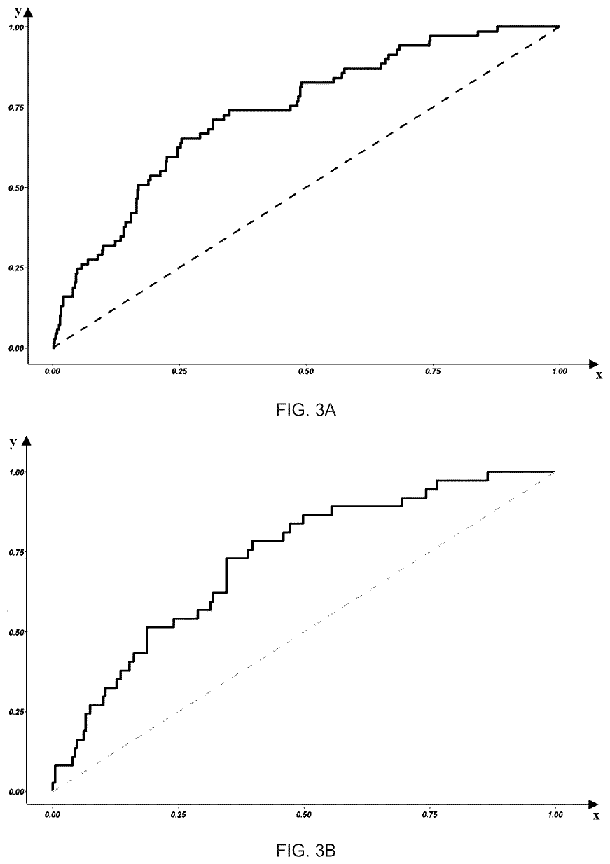

The present invention validates the constructed prediction model by generating receiver operating characteristic (ROC) curves based on the data from the training and validation sets, respectively, to evaluate sensitivity (i.e., true positive rate) and specificity (i.e., true negative rate). In FIGS. 3A and 3B, the x-axis represents the false positive rate (i.e., 1-specificity), and the y-axis represents the true positive rate (i.e., sensitivity). The area under the ROC curve (AUC), defined as the area enclosed by the ROC curve (solid line in FIGS. 3A and 3B) and the coordinate axes, is used to assess the quality of the risk nomogram in distinguishing true positives from false positives. The AUC values of the constructed prediction model are all above 0.6 (i.e., exceeding the area under the dashed reference line), specifically: 73.75% (95% CI: 67.72%-79.78%) for the training set (FIG. 3A), and 72.63% (95% CI: 64.36%-80.9%) for the validation set (FIG. 3B), indicating that the constructed model of the present invention demonstrates favorable robustness.

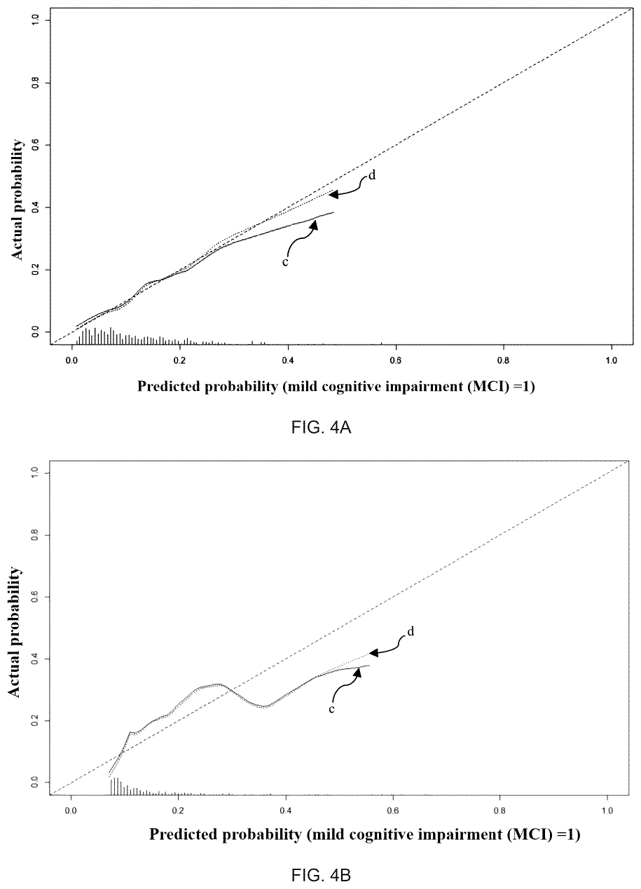

Additionally, calibration curves are used to evaluate the consistency between the predicted probabilities and the actual probabilities. The calibration curves for both the training and validation sets exhibit good consistency. As shown in FIGS. 4A and 4B, the dashed curve (as indicated by d) represents the actual observed probability of cognitive impairment, while the solid curve (as indicated by c) represents the probability predicted by the prediction model. These validation results collectively demonstrate that the cognitive impairment prediction model constructed in the present invention exhibits good predictive performance.

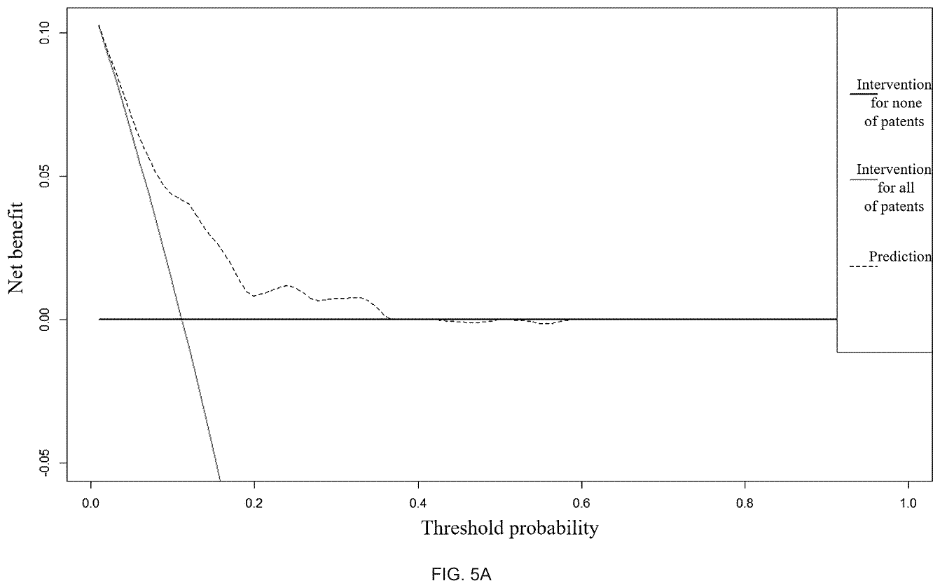

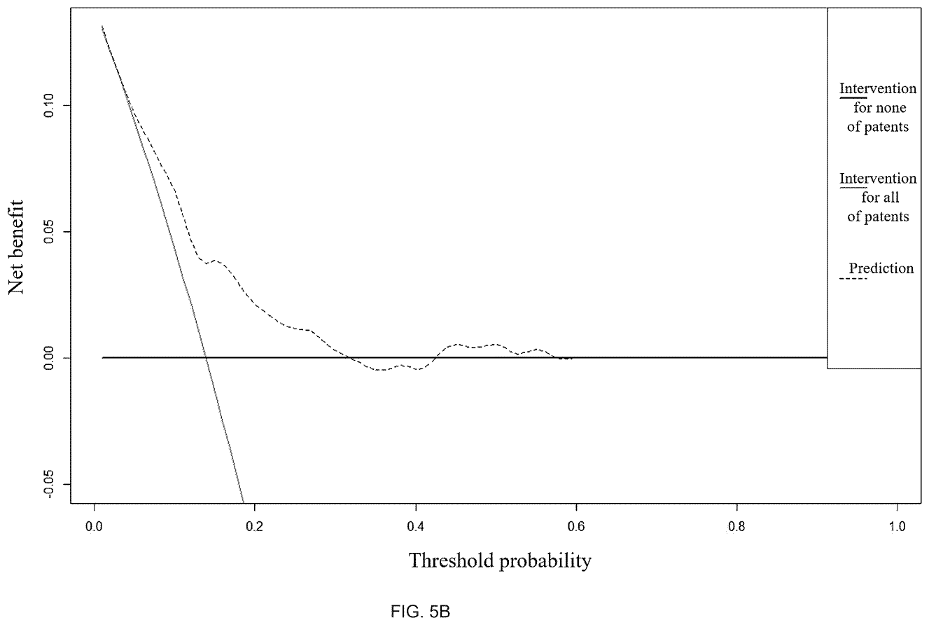

Furthermore, decision curve analysis (DCA) demonstrates that the effective threshold probability range for cognitive impairment risk prediction is 11% to 36% in the training set (FIGS. 5A) and 13% to 32% in the validation set (FIG. 5B). Notably, these ranges overlap between 13% and 32%. This overlapping range (i.e. the overlapped threshold probability range of the DCA curves between the training set and the validation set) indicates that, within this interval, both the training and validation sets yield a net positive benefit when individuals with predicted risk exceeding the threshold probability are classified as true positives and provided with interventions. In other words, individuals with a predicted probability greater than 0.32(32%) are considered to have a higher risk of developing cognitive impairment, and this threshold is clinically meaningful, as it is associated with a net benefit. It also demonstrates that setting the risk threshold within this overlapping range improves the practical utility of the prediction model, and the resulting benefit is quantifiable through the evaluation of net benefit.

### Embodiment 2: Predicting a Probability of a Subject Developing Cognitive Impairment Using the Above Cognitive Impairment Prediction Prediction Model

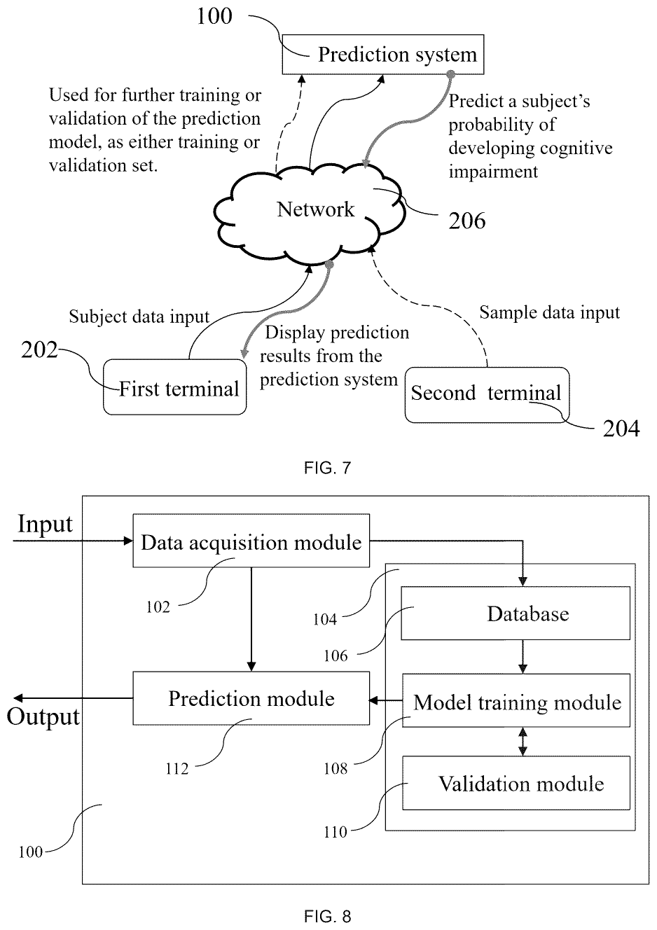

Referring to FIG. 7, FIG. 7 illustrates a schematic diagram of the optional architecture of the prediction system provided in the embodiment of the present invention. To support an exemplary application, terminals (exemplarily shows first terminal 202 and second terminal 204) are connected to the prediction system via a network. The network involved in the present system may be a wide area network (WAN), a local area network (LAN), or a combination thereof, and data transmission may be realized via a wireless link. The terminals involved in the present system may include various types of user terminals, such as smartphones, tablets, and laptop computers. The terminals can be used to display an interface for inputting subject data and/or sample data, as well as an interface for displaying prediction results from the prediction system.

The following describes an exemplary structure of the prediction system. In some embodiments, as shown in FIG. 8, a prediction system 100 may comprise:
a database 106 configured to store data, wherein a type of the data comprises gastrointestinal electrical signal data and clinical data, the data comprise sample data from a sample population and subject data from a subject;
a data acquisition module 102 configured to acquire the data and store the data in the database 106;
a model training module 108 configured to train the sample data using a machine learning algorithm to establish a prediction model (e.g., a cognitive impairment prediction model);
a prediction module 112 configured to acquire the subject data through the data acquisition module 102, and analyze the subject data using the established prediction model (e.g., the cognitive impairment prediction model) to predict a probability of the subject developing cognitive impairment;

The prediction system 100 may further comprise a validation module 110 configured to evaluate an accuracy of the established prediction model (e.g., the cognitive impairment prediction model), wherein an evaluation indicator of the evaluation comprises one or more of calibration, discrimination, and clinical utility.

The database 106, the model training module 108, and the validation module 110 may be integrated into a model construction module 104.

As an example, in the cognitive impairment prediction system of Embodiment 1, the gastrointestinal electrical signal data specifically include postprandial gastric dominant frequency, postprandial gastric percentage of disturbed electrical rhythm, postprandial gastric dominant power ratio, and postprandial intestinal lead time difference; and the clinical data specifically include age, body mass index (BMI), high-density lipoprotein, and low-density lipoprotein.

A specific application scenario of the present invention is provided below.

Community A conducts a large-scale screening activity for cognitive impairment, collecting gastrointestinal electrical signal data, blood glucose and blood lipid data, and individual characteristic data (the latter two collectively referred to as “clinical data”) from the target population within the community (e.g., middle-aged and elderly people). The subject data are entered via the first terminal 202. The subject data are transmitted via the network 206 to the data acquisition module 102 of the prediction system 100. The data acquisition module 102 acquires the subject data from a subject and stores them in the database 106.

The prediction module 112 acquires the subject data and analyzes the subject data using the established cognitive impairment prediction model to predict a probability of the subject developing cognitive impairment.

As an output, the prediction module 112 may generate a report indicating the subject's risk of developing cognitive impairment and transmit the prediction result to the first terminal 202 via the network 206. Community A may predefine a risk threshold for cognitive impairment (e.g., a probability of developing cognitive impairment probability of 30%). If a subject (e.g., patient B) is predicted to have a risk of cognitive impairment exceeding the risk threshold (e.g., a probability of 40%), Community A shall issue an alert to patient B or their family and recommend seeking further medical consultation at a recommended or partner hospital. When patient B visits the hospital, further and more comprehensive examinations are conducted to determine whether the patient is indeed suffering from the indicated cognitive impairment. The physician may transmit the diagnostic result of patient B to the prediction system 100 via the second terminal 204. The data of patient B (subject data +diagnostic result) can serve as new sample data for further training of the cognitive impairment prediction model. Of course, the diagnostic result of patient B may also be transmitted to the prediction system 100 via the first terminal 202. In other words, the terminal transmitting the diagnostic result may be the same or different.

From the above description of the embodiments, those skilled in the art will clearly understand that the methods described in the above embodiments can be implemented via software in combination with a necessary general-purpose hardware platform. Of course, they can also be implemented in hardware, although in many cases, the software-based implementation is preferred. Based on this understanding, the essence of the technical solution of the present invention—or the part that contributes to the prior art-may be embodied in the form of a software product. The computer software product is stored in a storage medium (such as ROM/RAM, magnetic disk, optical disk) and includes a set of instructions to cause a computer terminal (such as a mobile phone, computer, server, or network device) to execute the methods of the embodiments described in the present invention.

The embodiments of the present invention are described above with reference to the accompanying drawings, but the present invention is not limited to the aforementioned specific embodiments. The aforementioned embodiments are merely illustrative and not limiting. For those of ordinary skill in the art, many forms can be made under the teaching of present invention without departing from the spirit of the present invention and the scope of the claims, all of which shall fall within the protection scope of the present invention.

1. A cognitive impairment prediction system based on a gastrointestinal electrical signal, comprising:
a database configured to store data, wherein a type of the data comprises gastrointestinal electrical signal data and clinical data, and the gastrointestinal electrical signal data are postprandial gastrointestinal electrical signal data;
wherein the postprandial gastrointestinal electrical signal data comprise postprandial gastric dominant frequency, postprandial gastric percentage of disturbed electrical rhythm, postprandial gastric dominant power ratio, and postprandial intestinal lead time difference; wherein the clinical data comprise age, body mass index, high-density lipoprotein, and low-density lipoprotein; wherein the data comprise sample data from a sample population and subject data from a subject;
a data acquisition module configured to acquire the data and store the data in the database;
a model training module configured to train the sample data using a machine learning algorithm to establish a cognitive impairment prediction model;
a prediction module configured to acquire the subject data through the data acquisition module and analyze the subject data using the cognitive impairment prediction model to predict a probability of the subject developing cognitive impairment.

2. The system of claim 1, wherein the sample data are divided into a training set and a validation set in a ratio of 7:3.

3. The system of claim 1, wherein the gastrointestinal electrical signal data are simultaneously collected through leads respectively positioned at a gastric body, a gastric antrum, a lesser gastric curvature, a greater gastric curvature, an ascending colon, a transverse colon, a descending colon, and a rectum.

4. The system of claim 2, the system further comprises a validation module configured to evaluate an accuracy of the cognitive impairment prediction model using the validation set; wherein an evaluation indicator of the evaluation comprises one or more of calibration, discrimination, and clinical utility.

5. The system of claim 1, wherein the cognitive impairment is mild cognitive impairment.

6. A method for constructing a cognitive impairment prediction system based on a gastrointestinal electrical signal, wherein the cognitive impairment prediction system comprises a cognitive impairment prediction model, the method comprises:
S1 acquiring sample data from a sample population, wherein a type of the sample data comprises gastrointestinal electrical signal data and clinical data;
S2 performing a pre-training on the sample data to select a predictive variable, wherein the selected predictive variable comprises postprandial gastric dominant frequency, postprandial gastric percentage of disturbed electrical rhythm, postprandial gastric dominant power ratio, postprandial intestinal lead time difference, age, body mass index, high-density lipoprotein, and low-density lipoprotein;
S3 based on the selected predictive variable, training the sample data using a machine learning algorithm to establish the cognitive impairment prediction model, thereby constructing the cognitive impairment prediction system based on a gastrointestinal electrical signal.

7. The method of claim 6, wherein the sample data are divided into a training set and a validation set in a ratio of 7:3.

8. The method of claim 6, wherein the pre-training comprises a first-round variable selection and a second-round variable selection; wherein the first-round variable selection comprises LASSO regression analysis, and the second-round variable selection comprises logistic regression analysis and stepwise regression analysis.

9. The method of claim 7, the method further comprises S4 evaluating an accuracy of the cognitive impairment prediction model using the validation set, wherein an evaluation indicator of the evaluation comprises one or more of calibration, discrimination, and clinical utility.

10. The method of claim 6, wherein the pre-training comprises ridge regression or a random forest model.
